(19) United States
(12) Patent Application Publication
(10) Pub. No.: US 2017/0119362 A1
(43) Pub. Date: May 4, 2017

(54) Device and Method for Closure of Atrial Septal Defects

(71) Applicant: Michael R. Afremov, Minnetonka, MN (US)

(72) Inventor: Michael R. Afremov, Minnetonka, MN (US)

(21) Appl. No.: 15/201,950

(22) Filed: Jul. 5, 2016

Related U.S. Application Data
(63) Continuation of application No. 12/030,979, filed on Feb. 14, 2008, now abandoned.

(51) Int. Cl. A61B 17/00; A61B 17/12

(52) U.S. Cl. CPC A61B 17/0057; A61B 17/12136; A61B 17/12181; A61B 2017/00575; A61B 2017/00606; A61B 2017/00623; A61B 2017/00004; A61B 17/12113; A61B 17/12122

(57) ABSTRACT

The present invention is directed towards implantable, inflatable, bioabsorbable medical prostheses. In particular, the present invention relates to an implantable, inflatable, bioabsorbable method and device for occluding septal defects such as an atrial septal defect. A double button shaped device is contained in a catheter to allow for easy positioning and re-positioning of the apparatus to ensure proper placement and deployment. The device is charged with a filling solution so that it temporarily stabilizes the defect for a period of time typically varying from weeks to a year while it provides a structure to support natural tissue growth. The device is eventually replaced by natural tissue as it degrades and is absorbed or eliminated from the body by natural processes.

### BACKGROUND OF THE INVENTION

The present invention relates generally to implantable, inflatable, bioabsorbable medical prostheses. In particular, the present invention relates to an implantable, inflatable, bioabsorbable method and device for occluding septal defects such as an atrial septal defect.

Septal defects generally refer to a perforation or hole passing through a septum. A septum is a thin wall of muscle separating two cavities. Atrial septal defects (ASD's) are a common congenital cardiac abnormality. A large atrial septal defect can lead to increased risk of heart attack, migraine headaches, enlargement of the right atrium and right ventricle among other circulatory and respiratory problems.

Some ASD's may be managed through pharmacological therapy which often includes oral anticoagulants or antiplatelet agents. These therapies may lead to certain side effects, including hemorrhage. If pharmacologic therapy is unsuitable, open heart surgery may be employed to close a ASD with stitches, for example. Like other open surgical treatments, this surgery is highly invasive, risky, requires general anesthesia, and may result in lengthy recuperation.

To close an ASD, open heart surgery has been used for decades. In such an operation, the patient's chest must be opened and heart temporarily bypassed. Then, the surgeon sutures the defect shut or if the defect is too large, a patch of material is sewn in place to close the aperture.

In order to avoid the trauma and complications caused by open heart surgery, transcatheter techniques to close septal defects have been attempted. These techniques deliver an occlusion device through a catheter to the septal defect. The device is placed into the defect and permanently deployed. Prior art devices include self-expanding umbrellas that are fixed to either side of the opening, double-disk devices made of self-expanding nitinol with Dacron fabric and clam-shell devices, among others.

The double-disk nitinol device is the most prevalent minimally invasive device used. There are known problems with the use of the prior art devices. A significant problem with the prior art devices is due to the necessity of leaving a relative large mass of nitinol in the heart. The potential erosion problems associated with the large mass include perforation, heart block and other complications up to and including death. Some of the problems relate to the fact the device is a permanent implant and as a treatment for congenital defects, it is often used on fairly young patients. Therefore the device is in place for very long period of time. Therefore these prior art devices require patients to participate in long-term routine follow-up doctor visits to monitor the placement and condition of the device.

Nitinol based prior art devices contain heavy metals including nickel which may, over the long term, produce toxicity problems in some individuals.

The prior art devices are generally held in place by clamping around heart tissue. With time the prior art device may erode tissue resulting in irritation and injury to the tissue and possible dislodgement of the device within the heart. Additionally many of these conventional devices used for ASD's, however, are technically complex, bulky, and difficult to deploy in a precise location.

Furthermore, due to the mass associated with the prior art devices; they are generally not suitable for use for occluding ASD's with large openings. An ASD greater than 40 mm will generally require closure through open-heart surgery simply due to the large mass that would be associated with a device required to close a larger ASD.

### BRIEF SUMMARY OF THE INVENTION

The invention includes a device and method for occluding abnormal apertures or openings in body walls or membranes. The device is adapted to be delivered through the body through conventional minimally invasive surgical techniques using a catheter system to the opening. One such abnormal opening which this invention is intended to occlude is an atrial septal defect. This invention may also be used to occlude other abnormal openings such as ventricular septal defects, patent foreman ovale, patent ductus arteriosus, aneurysms in blood vessels or other similar body lumens.

The term “bioabsorbable,” as used in this application, is also understood to refer to materials that are at least partially degradable through enzymatic or hydrolytic action so that over time they will structurally degrade and may be substantially eliminated from the body through conventional natural oxidation or elimination processes.

In this application, “distal” refers to the direction away from a catheter insertion location and “proximal” refers to the direction nearer the insertion location.

The device is delivered through a catheter that allows for easy positioning and re-positioning of the apparatus to ensure proper placement and deployment. The device is charged with a filling solution so that it temporarily stabilizes the defect for a period of time while it provides a structure to support natural tissue growth. With sufficient time, the tissue growth will completely cover the device and will be sufficient to occlude the defect by itself. The device is designed so that it eventually replaced by natural tissue as the device degrades into byproducts which can then be absorbed or eliminated from the body by natural processes.

Prior to use, the device resembles an empty bladder or membrane which is arranged for use to fit within a catheter for transport to the defect. The membrane is made predominately of a bioabsorbable polymer or collagen-based materials with the appropriate tensile strength, service life and other properties preferred for the particular application.

The empty membrane incorporates or is attached to a fastener. The fastener can then be releasably connected to an insertion cannula which is disposed within a catheter lumen. The insertion cannula may be reinforced with a winding to provide extra support to push the device through the catheter. Following positioning of the catheter at the ASD, the insertion cannula is used to displace the device from the catheter and properly position the device for inflation. The insertion cannula preferably encloses a fluid conduit for carrying a filling solution into the device which may be of a wire or cable type fabrication. Although the insertion cannula preferred embodiment is to include the pusher function and the filling function, under alternative embodiments it is possible to use a separate element for each of these functions. The filling solution is preferably a saline, contrast solution, or a radiopaque solution depending on the preferred properties for the specific patient.

Filling the device provides the internal mass to generate the desired final shape of the device from the empty membrane. The filling solution also aids in providing structural integrity to the device for proper placement within the ASD opening. Upon filling, the membrane preferably forms into a double button form with a central connector between a proximal lobe and a distal lobe. The device is fabricated and made available with a variety of sized central connectors, in 1 to 2 millimeter increments, so that the central connector can be chosen that is approximately the same diameter, or slightly larger than the diameter of the ASD opening.

Since atrial blood pressure is somewhat higher on the left atrium than within the right atrium, the distal lobe is preferably a slightly larger diameter than the proximal lobe to help prevent dislodgement of the device. Through manipulation of the catheter, the central connector is positioned within the ASD opening with the proximal and distal lobes transposed on opposing sides of the septa.

Placement of the device may be accomplished by pushing the distal lobe portion out of the catheter, positioning the device so that the distal lobe portion is in the left atrium and the central connector is positioned within the defect. The device is then partially inflated and the distal lobe is brought up against the septum. The proximal lobe portion is then pushed from the catheter and as or just after it is released from the catheter it is also inflated. Alternatively, the device can be pushed completely from the catheter in one step, positioned within the defect and then inflated as a unit complete unit.

The deployment of the device can be examined through various techniques. If necessary, the device can be easily deflated and repositioned, or removed and replaced with a different device. Once proper operation of the device is confirmed, the device is then detached from the insertion cannula and the catheter and insertion cannula are removed from the patient. The device preferably includes a self sealing valve to maintain the filling solution within the membrane after detachment of the device from the insertion cannula.

Placement of the device within the defect, allows the membrane to provide a temporarily stabile surface to allow tissue growth through natural body repair processes. The membrane is designed to degrade into products that can be eliminated by natural body processes after its initial stabilization function is no longer needed. The membrane surfaces may be coated or conditioned with various treatments to achieve beneficial therapeutic effects such as to promote occlusion, thrombosis and initiate formation of the new living tissue to replace the material of the device as it biodegrades.

Due to the low mass and bioabsorbable properties of the device, the device can be used to effectively repair larger ASD's than prior art devices. In addition, the device reduces the need of a patient to have long term follow up medical examinations to monitor position of the implanted foreign mass, device toxicity or erosion problems associated with a permanent metal mass in the heart since these issues are not a consideration with a bioabsorbable device that does not remain in the patient's body long-term.

### BRIEF DESCRIPTION OF THE DRAWINGS

FIG. 1 is an illustration of the device as transported in a catheter.

FIG. 2 is an illustration of the device in an intermediate partially filled state prior to use.

FIG. 3 is an illustration of the device in its collapsed state prior to use.

FIG. 4 is an illustration of an embodiment of the device positioned in an opening.

FIG. 5 is an illustration of an expanded view of one embodiment of the device attached to a septal opening.

FIG. 6 is an illustration of an expanded view of another embodiment of the device attached to a septal opening

FIG. 7 is an illustration of an expanded view of one embodiment of the fastener portion of the device.

FIG. 8 is an illustration of an expanded view of another embodiment of the fastener portion of the device.

### DETAILED DESCRIPTION OF THE INVENTION

The invention includes a device and method for occluding apertures or openings in body walls or membranes. The device is adapted to be delivered through the body by a catheter system to the abnormal opening. One such abnormal opening which this invention can occlude is an atrial septal defect (ASD). Atrial septal defect is a common congenital cardiac abnormality that is the type of abnormal opening for which the preferred embodiments of the invention are designed, but this invention may be used to occlude other abnormal openings such as ventricular septal defects, patent foreman ovale, patent ductus arteriosus, aneurysms in blood vessels, vascular plug or other similar body lumens.

Prior to deploying the device 10 the size of the opening is determined so that the appropriate sized device 10 may be selected. The opening may be sized by using conventional imaging techniques or inserting a balloon catheter into the opening and inflating the balloon to determine the opening size.

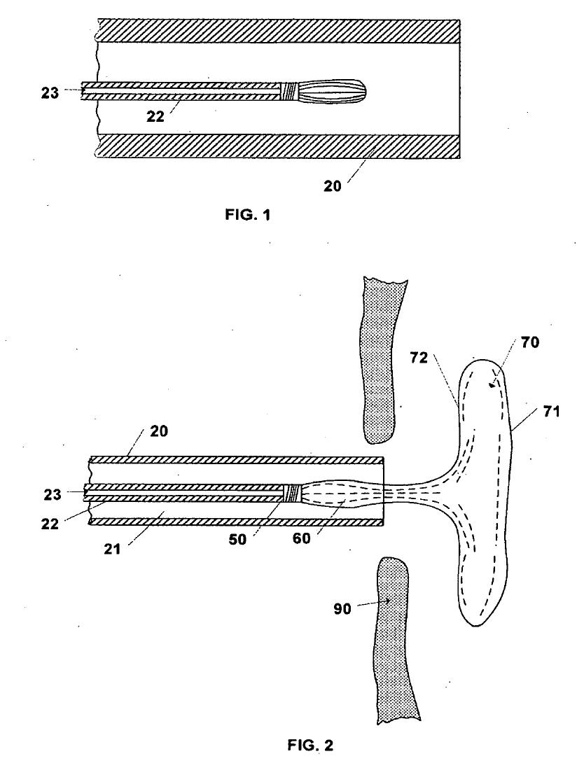

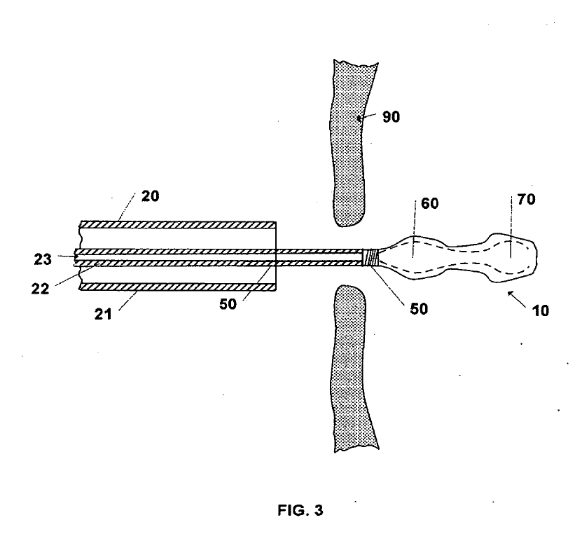

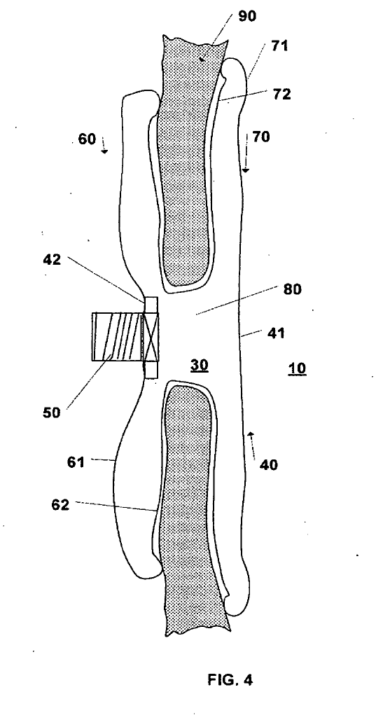

FIGS. 1-4 show the device 10 at various stages of deployment. FIG. 1 shows the device 10 folded into the catheter 20 for transport to the defect site. FIGS. 2 and 3 show the device 10 attached to the insertion cannula 22 prior to complete filling being expelled from the catheter 20 in position near the septal defect. FIG. 4 shows the device 10 after filling and after detachment from the insertion cannula 22.

The device 10 is adapted to be delivered using standard minimally invasive surgical techniques through the patient's body by a catheter system to the abnormal opening. The device 10 is contained in a catheter 20 to allow for easy positioning and re-positioning of the apparatus to ensure proper placement and deployment. The device 10 is charged with a filling solution 30 after which the device 10 will stop the blood flow within minutes. The device 10 then temporarily stabilizes the defect for a period of time typically varying from days to over a year while it provides a structure to support natural tissue growth. The device 10 is fabricated from a material designed to start dissolving within a predetermined amount of time which may vary from hours to as long as 1 or 2 years. The device 10 is eventually totally replaced by natural tissue as it degrades and is absorbed or eliminated from the body by natural processes. During the period of its degradation, it is important that the device does not break into large particles that may freely pass into the blood stream which could result in strokes or other blockages.

Prior to use, the device 10 resembles an empty bladder or membrane 40 which is arranged for use to fit within a catheter 20 for transport to the defect. The membrane is made predominately of a bioabsorbable polymer or collagen-based materials with the appropriate tensile strength a structural stability for the particular application.

The empty membrane 40 is attached to a fastener 50 that can be releasably connected to an insertion cannula 22 which is disposed within the catheter lumen 21. The empty membrane 40 may also be encapsulated within a carrier catheter or other sleeve to prevent any binding and to assist movement of the device through the catheter lumen 21 to the defect. Following positioning of the catheter 20 at the ASD, the insertion cannula 22 is used to displace the device 10 from the catheter 20 and properly position the device 10 for inflation. The insertion cannula 22 may be of a wire or cable type fabrication with or without wire wound reinforcement. The insertion cannula 22 preferably encloses a fluid conduit 23 to transport a filling solution 30 into the device 10 to fill the membrane 40. Although the insertion cannula 22 preferred embodiment is to include the pusher function and the filling function, under alternative embodiments it is possible to use a separate element for each of these functions. Under the preferred embodiment, fluid communication between the fluid conduit 23 and the membrane 40 is through the fastener 50. The fastener 50 may be threaded or selected from other releasable mechanical connectors known in the prior art. The filling solution 30 is preferably a saline or contrast solution depending on the particular application.

The device 10 is filled to provide structural integrity for placement within the ASD opening. Upon filling in one embodiment, the membrane transposes into a double button form with a central connector 80 between a proximal lobe 60 and a distal lobe 70. The proximal lobe 60 may include a proximal lobe cap 61 and a proximal lobe retention surface 62. The distal lobe 70 may contain a distal lobe cap 71 and a distal lobe retention surface 72. Since atrial blood pressure is higher on the left atrium than on the right atrium, the distal lobe 70 is preferably a slightly larger diameter than the proximal lobe 60, as shown in FIG. 4. This helps to prevent dislodgement of the device 10 from the ASD. The central connector 80 of the device 10 is positioned within the ASD opening with the proximal and distal lobes transposed on opposing sides of the heart tissue 90 surrounding the septa. The device 10 is fabricated with a variety of sized central connectors 80, in 1 to 2 millimeter increments, so that the central connector 80 can be chosen that is approximately the same diameter, or slightly larger than the diameter of the ASD opening.

The device 10 is then detached from the insertion cannula 22 and the catheter 20 and insertion cannula 22 are removed from the patient. The device 10 preferably includes a valve 42 which may be composed of a self sealing material or a check valve to maintain the filling solution 30 within the membrane 40 after detachment of the device 10 from the insertion cannula 22.

The membrane 40 provides a temporarily stabile surface to allow tissue growth through natural body repair processes. The membrane 40 is designed to degrade into products that can be eliminated by natural body processes after its stabilization function is no longer needed. The membrane surfaces may have a membrane coating 41 or conditioned with various treatments to achieve beneficial therapeutic effects. For example, it may be desirable to enhance the roughness of portions of the device surface to promote thrombosis and angiogenesis thereby increasing the rate of tissue growth.

The device 10 itself is pliable due to the tensile strength of the polymeric material from which its walls are constructed, but becomes rigid when filled with the chosen liquid or gel. Thus, upon inflation, the present invention is able to exert radial force against the walls to which it is deployed and able to resist compression.

FIG. 1 generally shows the device 10 contained within the catheter 20 for the delivery, deployment and positioning process. The device 10 is delivered in proximity to the opening site. Once the catheter 20 has been positioned, the device 10 is advanced past the distal end of the catheter 20 by advancing the catheter lumen 21 and device 10 as a unit so that the distal lobe portion is disposed outside of the catheter 20, positioning the device so that the distal lobe 70 portion is in the left atrium and the central connector 80 is positioned at the distal end of the catheter 20. As shown in FIG. 2, the device 10 is then partially inflated and the distal lobe 70 is positioned against the septum. The proximal lobe 60 portion is then pushed from the catheter 20 and as it is released from the catheter 20, after which it is also inflated.

Alternatively, as shown in FIG. 3, the device 10 can be pushed from the catheter 20, positioned within the defect and then inflated as a unit. Once the device 10 is properly positioned, the catheter lumen 21 is used to remove the device 10 from the catheter 20 to a position similar to that shown in FIG. 3. The catheter 20 withdrawal is achieved by removing the catheter 20 to a calibrated distance marked on a proximal end of the catheter 20.

During placement, some filling solution 30 such as saline or contrast solution may be introduced to the device 10 so that the position of the device 10 may be verified either angiographically or by transesopshageal echocardiography. If the position and/or size is not as desired, the entire device 10 can be removed from the opening by removing the filling solution 30 and withdrawing the device 10 from the defect and replacing and/or repositioning the device 10. If the device 10 is properly positioned and correctly sized, the device 10 may be filled to a final size and placement. The device 10 is then disengaged from the insertion cannula 22 and the catheter 20 and insertion cannula 22 may be removed. Once disengaged, the entire delivery system is withdrawn from the opening with the occlusion device left remaining in place to occlude the opening.

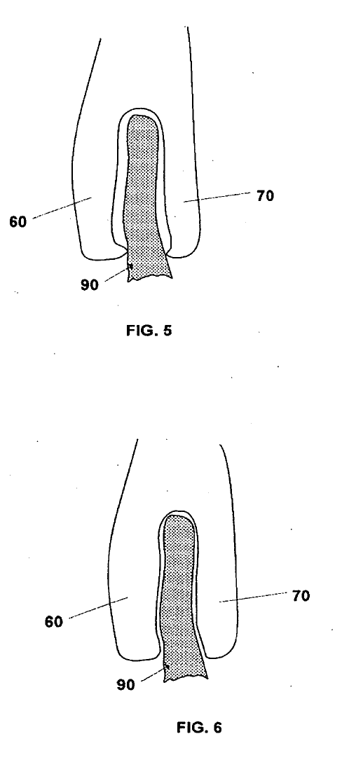

FIGS. 5 and 6 show expanded views of alternative geometries for the distal and proximal lobes surfaces for engaging the heart tissue 90.

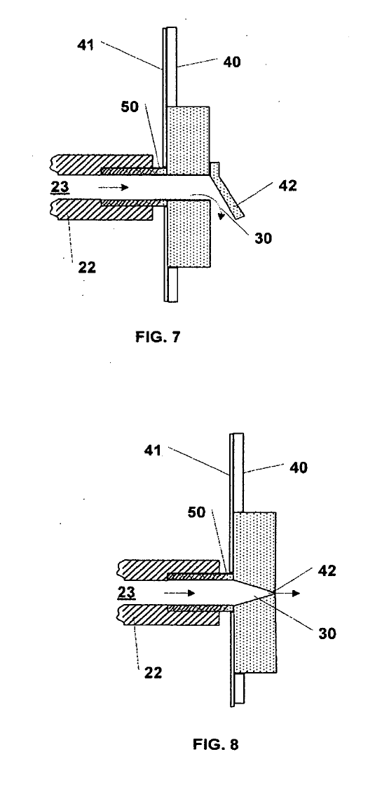

As shown in FIGS. 7 and 8, a valve 42, also dissolvable and preferably of the same material used in the membrane 40 may be employed to prevent deflation of the device 10 after deployment. Other mechanisms, including valve-like mechanisms made of bioabsorbable polymer may also be used for the purposes of the present invention. Examples include a duck-bill valve, flap valve, self-sealing material or other known medical device valves. Such mechanisms, including those with elastic self-sealing properties such as shown in FIG. 8, are well known in the art. Alternatively, mechanisms which detach with heating of a platinum electrode wire may be used to seal the inflated device 10.

In preferred embodiments of the invention, inflation of the device 10 takes place in situ, i.e. after the device 10 is deployed to its desired location. The filling solution 30 can include fluid, gel-like, liquid, gaseous, or solid-phase compositions (i.e. for example lyophilized material or nanoparticles), as well as combinations of such compositions. This allows for the deployment of the device 10 in a substantially compressed form and permits device 10 placement by minimally invasive techniques.

In preferred embodiments, the device 10 is preferably made of a bioabsorbable polymer or collagen based material with the appropriate tensile strength to withstand insertion into and removal from a catheter 20, expansion and stretching as the device 10 is filled, placement at the appropriate location within the patient and stresses imposed by the localized environment within the patient for its lifetime of the particular application.

Examples of polymers suitable for the purposes of the present invention include biodegradable polymeric compounds, including polymers of lactic acid, poly(alpha-hydroxy acid) such as poly-L-lactide (PLLA), poly-D-lactide (PDLA), polyglycolide (PGA), polydioxanone, polyglycolic acids, polycaprolactone, polygluconate, polylactic acid-polyethylene oxide copolymers, modified cellulose, collagen, poly(hydroxybutyrate), polyanhydride, polyphosphoester, poly(amino acids), tyrosine-derived polycarbonates, poly-lactic-co-glycolide (PLGA) or related copolymers, as well as blends of the foregoing polymers, or their respective monomers, dimers, or oligomers, each of which have a characteristic degradation rate in the body. For example, PGA and polydioxanone are relatively fast-bioabsorbing materials (weeks to months) and PLA and polycaprolactone are a relatively slow-bioabsorbing material (months to years). All of these materials are readily available and well known to a person of skill in the art, with HDPE being the preferred bio-material of use in the ASD device.

Suitable collagen-based materials have been manufactured and disclosed in the literature. Collagen-based materials are desirable in view of their biocompatibility, resorbability properties. Cohesive films of high tensile strength have been manufactured using collagen molecules or collagen-based materials.

When used in an occlusion device 10, bioabsorbable material can assist natural tissue regrowth which may include: (1) stimulation in the infiltration of native cells into an acellular matrix; (2) stimulation of new blood vessel formation (capillaries) growing into the matrix to nourish the infiltrating cells (angiogenesis); and/or (3) effecting the degradation and/or replacement of the bioabsorbable material by endogenous tissue upon implantation into a host.

This arrangement allows for the selective release of one drug toward the heart tissue against which the device 10 rests and the selective release of another drug to the atrial chambers on the opposite side.

In a further embodiment of the present invention, the device membrane 40 may additionally be coated with a drug or therapeutic agent. Alternatively, or additionally, the composition of choice may be embedded in the polymeric device membrane 40 or covalently bound to it by processes well known in the art. Such compositions of choice may include anticoagulants, antimicrobials, chemoattractants, chemotherapeutics, i.e. angiopeptin, methotrexate, heparin, as well as drugs that positively affect healing at the site where the device is deployed, either incorporated into the polymer forming the device, or incorporated into the coating, or both. Other suitable drugs may include antithrombotics (such as anticoagulants), antimitogens, antimitotoxins, antisense oligonucleotides, gene therapy vehicles, nitric oxide, and growth factors and inhibitors. Known direct thrombin inhibitors include hirudin, hirugen, hirulog, PPACK (D-phenylalanyl-L-propyl-L-arginine chloromethyl ketone), argatreban, and D-FPRCH2 Cl (D-phenylalanyl-L-propyl-L-arginyl chloromethyl ketone); indirect thrombin inhibitors include heparin and warfarin. All of these compositions preferably are incorporated in quantities that permit desirable timed release as the device and/or coating biodegrades.

The Tillable portion of the device 10 may also be inflated with drugs that can help dissolve plaque, act as anticoagulants to prevent distal emboli or chemoattractants to promote infiltration/recruitment of stem cells to site of injury. One advantage of the design of the present invention is the ability to deliver much larger quantities of therapeutic compositions to locations of choice, as the device filling solution 30 is able to accommodate a significant amount of material as compared to the more limited ability of a device coat to accommodate therapeutic agents. Material selection of the membrane may be optimized to allow the drug to permeate through the membrane if preferred. The amount of drug or therapeutic composition that can be delivered, as well as the time over which it is delivered, are thus vastly increased by device of the present invention.

In some embodiments, to prevent the sharp edges of the lobe portions of the occlusion device from causing tissue damage, proximal lobe retention surface 62 and the distal lobe retention surface 72 the lobe edges may be arranged to have a bulbous profile. In other words, the edge portion of at least one lobe is configured to have a smooth profile and form a bulge in the direction opposite of the lobe center. FIG. 3 is a side view diagram illustrating the contour of an embodiment of an occlusion device having enlarged, bulbous lobe edges. This arrangement allows more force to be exerted in the direction of the septum to more securely hold the device 10 in place. In addition, the proximal lobe cap 61 may have a concave portion to avoid a protuberance from the fastener and to promote tissue growth over the attachment point defined by the fastener.

Having described preferred embodiments of the invention, it should be apparent that various modifications may be made without departing from the spirit and scope of the invention, which is defined in the claims below.

It should be appreciated that elements described with singular articles such as “a”, “an”, and/or “the” and/or otherwise described singularly may be used in plurality. It should also be appreciated that elements described in plurality may be used singularly.

Although specific embodiments of apparatuses and methods have been illustrated and described herein, it will be appreciated by those of ordinary skill in the art that any arrangement, combination, and/or sequence of that is calculated to achieve the same purpose may be substituted for the specific embodiments shown. It is to be understood that the above description is intended to be illustrative and not restrictive. Combinations of the above embodiments and other embodiments as well as combinations and sequences of the above methods and other methods of use will be apparent to individuals possessing skill in the art upon review of the present disclosure.

The scope of the claimed apparatus and methods should be determined with reference to the appended claims, along with the full scope of equivalents to which such claims are entitled.

1. A method for occluding an opening within a body surface comprising the steps of:
a) providing a device having a membrane attached to a fastener oriented opposite a central connector, the membrane also having a proximal lobe adjoining the fastener and a distal lobe, the proximal lobe connected to the distal lobe by the central connector;
b) affixing an insertion cannula to the fastener of the device;
c) inserting the device into the catheter;
d) transporting and positioning the device to the opening;
e) advancing the device out of the catheter;
f) filling the membrane with a filling solution;
g) occluding the opening;
h) disengaging the device from the insertion cannula from the fastener of the device; and
i) withdrawing the catheter and insertion cannula.

2. The method of claim 1 wherein prior to the step of occluding the opening the device may be repositioned within the opening.

3. The method of claim 1 having the additional step of applying a therapeutic agent to the membrane.

4. The method of claim 1 wherein the membrane is composed of a biodegradable material.

5. The method of claim 1 wherein the membrane is composed of a biodegradable high density polyethylene.

6. The method of claim 1 wherein the filling solution contains a radiopaque material.

7. The method of claim I wherein the step of occluding the opening includes securing a septa between the proximal lobe and the distal lobe.

8. The method of claim 1 wherein the opening has a maximum size greater than 40 millimeters.

9. A method for occluding an opening within a body surface comprising the steps of:
a) providing a device having a membrane attached to a fastener oriented opposite a central connector, the membrane also having a proximal lobe adjoining the fastener and a distal lobe, the proximal lobe connected to the distal lobe by the central connector;
b) affixing an insertion cannula to the fastener of the device;
c) inserting the device into the catheter;
d) transporting and positioning the device to the opening;
e) advancing the distal lobe out of the catheter;
f) filling the distal lobe with a filling solution;
g) engaging the distal lobe with the opening;
h) advancing the proximal lobe out of the catheter;
i) filling the proximal lobe with a filling solution;
j) disengaging the device from the insertion cannula from the fastener of the device; and
k) withdrawing the catheter and insertion cannula.

10. A bioabsorbable device comprising:
a) a membrane attached to a fastener oriented opposite a central connector, the membrane also having a proximal lobe adjoining the fastener and a distal lobe, the proximal lobe connected to the distal lobe by the central connector;
b) wherein the membrane is inflatable with a filling solution.

11. The device of claim 9, wherein the membrane provides structural rigidity when inflated.

12. The device of claim 9, wherein the membrane the filling solution contains a therapeutic agent.

13. The device of claim 9, wherein the membrane is coated with a therapeutic agent.

14. The device of claim 9, wherein the membrane is inflated with a contrast agent.

15. The device of claim 9 wherein the membrane is composed of a biodegradable material.

16. The device of claim 9 wherein the membrane is composed of a biodegradable high density polyethylene

17. The device of claim 9 wherein the central connector expands to fill the opening.

18. The device of claim 9 wherein the device is positioned in a catheter.

19. The device of claim 9 wherein the fastener is attached to a catheter lumen.

20. The device of claim 9 also having a septa is positioned between the proximal lobe and the distal lobe wherein the distal lobe is larger than the proximal lobe.

21. (canceled)
